(19) United States
(12) Patent Application Publication
(10) Pub. No.: US 2024/0358629 A1
(43) Pub. Date: Oct. 31, 2024

(54) SELF-PROPELLED PHARMACEUTICAL DELIVERY CAPSULES

(71) Applicant: Cochlear Limited, Macquarie University, NSW (AU)

(72) Inventors: Wolfram Frederik DUECK, Berlin (DE); Daniel SMYTH, Dublin (IE)

(21) Appl. No.: 18/577,485

(22) PCT Filed: Jun. 21, 2022
(86) PCT No.: PCT/IB2022/055749

§ 371 Date: Jan. 8, 2024

Related U.S. Application Data
(60) Provisional application No. 63/223,198, filed on Jul. 19, 2021.

(51) Int. Cl. A61K 9/46; A61K 9/00; A61K 31/573

(52) U.S. Cl. CPC A61K 9/0007; A61K 9/0046; A61K 31/573

(57) ABSTRACT

Pharmaceutical compositions and methods of their administration following delivery of such compositions into a fluid-containing chamber of the body are provided. More specifically, pharmaceutical compositions mixed with reactive compositions produce a gas when brought into contact with a bodily fluid, such as that in the cochlear canal; the gas causes a fluidic movement in the chamber and also propels the disintegrating composition containing the therapeutic substance in the fluid to the apical region of the canal where the therapeutic substance contacts the tissue walls of the chamber, leading to various effects on or in cells of the tissue surrounding the fluid in that region. Methods disclosed may be used for delivery of pharmaceutical compositions to other fluid-containing chambers of the body for treatment of tissues in difficult-to-reach areas, and may be especially useful in chambers in which there is limited fluid movement.

### BACKGROUND

### Field

Presented herein are pharmaceutical compositions and methods of their administration in fluid-containing compartments/chambers of the body.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a pharmaceutical delivery capsule configured for delivery of a therapeutic substance to a fluid-containing chamber of the body is provided. The pharmaceutical composition comprises an effective dose of the therapeutic substance; and a pharmaceutically acceptable reactive composition that generates a gas upon contact with a bodily fluid in said fluid-containing chamber, wherein said pharmaceutical composition and said reactive composition are disposed within a permeable or semi-permeable coating layer that permits diffusion of aqueous fluid through the coating layer to contact said reactive composition, and wherein said reactive composition comprises one or more channels that allow escape of said gas from said capsule thereby propelling said capsule within the fluid-containing chamber.

In another aspect, a pharmaceutical composition configured for delivery of a therapeutic substance to a fluid-containing chamber of the body is provided. The pharmaceutical composition comprises: an effective dose of the therapeutic substance; and a pharmaceutically acceptable reactive composition that generates a gas upon contact with a bodily fluid in said fluid-containing chamber, wherein the pharmaceutical composition and the pharmaceutically acceptable reactive composition are each in a form selected from the group consisting of a liquid, a powder, a particulate, and a gel, and wherein the generation of the gas upon contact with the pharmaceutically acceptable reactive composition with a bodily fluid creates motion in the fluid-containing chamber, distributing the therapeutic substance throughout the fluid-containing chamber

In another aspect, a method of administering a therapeutic substance to a fluid-containing chamber of the body is provided. The method comprises: contacting said chamber with a pharmaceutical delivery capsule comprising: a pharmaceutical composition comprising an effective dose of the therapeutic substance; a pharmaceutically acceptable reactive composition that generates a gas upon contact with a bodily fluid in said fluid-containing chamber, wherein said pharmaceutical composition and said reactive composition are disposed within a permeable or semi-permeable coating layer that permits diffusion of aqueous fluid through the coating layer to contact said reactive composition, and wherein said reactive composition comprises one or more channels that allow escape of said gas from said capsule thereby propelling said capsule within the fluid-containing chamber.

In another aspect, a method of administering a therapeutic substance to a fluid-containing chamber of the body is provided. The method comprises: contacting said chamber with a pharmaceutical composition, wherein the pharmaceutical composition comprises: an effective dose of the therapeutic substance; and a pharmaceutically acceptable reactive composition, wherein said pharmaceutical composition and said reactive composition are mixed and administered together, and upon contact with said bodily fluid, generate gas, and wherein said gas increases movement of the fluid in the fluid-containing chamber and leads to distribution of the therapeutic substance and subsequent contact of the therapeutic substance with barrier tissues of the fluid-containing chamber.

In another aspect, a method for treating a patient is provided. The method comprises: administering a pharmaceutical composition comprising a therapeutic substance directly into a basal region of a cochlear canal of the patient to contact fluid in said basal region, wherein said fluid in said cochlear canal extends into an apical region of said canal, wherein said pharmaceutical composition further comprises a reactive composition which, when in contact with said fluid, generates a gas, which leads to distribution of said therapeutic substance throughout said fluid in said cochlear canal, and wherein said distribution permits contact with and uptake of said therapeutic substance by tissues surrounding said fluid throughout the cochlear canal.

### BRIEF DESCRIPTION OF THE DRAWINGS

Embodiments disclosed are described herein in conjunction with the accompanying drawings, in which:

FIGS. 1A and 1B show diagrams of the middle and inner ear anatomy of a human.

FIG. 2 is a schematic diagram illustrating a self-propelled capsule, in accordance with certain embodiments presented herein.

FIG. 3 is a schematic diagram illustrating another self-propelled capsule, in accordance with certain embodiments presented herein.

FIGS. 4A, 4B, and 4C shows a series of photographs depicting a before, start, and after 3 min results for a brief experiment for (1) pure ink; and (2) ink added to a powder mixture of sodium bicarbonate, diphosphate and corn starch; added to physiological saline solution (0.9% NaCl) contained in a plastic tube. Results shown in the photographs reveal that the ink when added in the presence of a powder mixture of sodium bicarbonate (CO₂ source), diphosphate (acid) and corn starch (separating agent) is distributed faster throughout the saline in three minutes than when the ink alone is present.

FIGS. 4A, 4B, and 4C are a series of photographs aspects of an experiment demonstrating distribution of ink alone in physiological saline solution, in accordance with certain embodiments presented herein;

FIGS. 5A, 5B, and 5C are a series of photographs aspects of an experiment demonstrating more rapid distribution of ink when present with a propellant, in accordance with certain embodiments presented herein;

FIG. 6 shows a flow chart for general process of administration of a therapeutic substance, in accordance with certain embodiments presented herein;

FIG. 7 is a flow chart of an example method, in accordance with certain embodiments presented herein; and

FIG. 8 is a flow chart of another example method, in accordance with certain embodiments presented herein.

### DETAILED DESCRIPTION

A growing area of research and development relates to the use of pharmaceutical compounds, biological substances, bioactive substances, etc., including pharmaceutical agents/active pharmaceutical ingredients (APIs), genes, messenger RNA (mRNA) or other signalling compounds that promote recovery and resolution, chemicals, ions, drugs, etc. to treat a variety of disorders within the body of individual patient/recipient. These various substances, which are collectively and generally referred to herein as “therapeutic substances,” are delivered to induce some therapeutic results/treatment within the body of the recipient. For example, therapeutic substances may be delivered to treat ear disorders (e.g., tinnitus, hearing loss, tinnitus, Ménière's disease, etc.), to treat infections post-surgery, to fight cancer cells, to treat neurodegenerative diseases, to treat infectious diseases, etc.

The body of an animal, including the body of a human recipient (“recipient”), includes a number of different body chambers. In certain examples, these body chambers are fluidically-sealed (e.g., cavities or enclosed areas in which bodily fluids are sealed). For example, sensitive tissues in the body of a recipient, such as the brain, the ear, the eye, etc. are protected from the normal circulation by fluidic tissue barriers. In particular, the brain is surrounded by the blood-brain barrier (BBB), the inner ear (including the cochlea and the vestibular system) are surrounded by the blood-labyrinth barrier (BLB), the eye retina is surrounded by the blood-ocular barrier (BOB), which includes the blood-aqueous barrier (BAB) and the blood-retinal barrier (BRB), and so on. Other tissue barriers, such as the round window, and/or the oval window, are also present in the body of a recipient and are two tissue barriers associated with a fluidically-sealed cochlea of a recipient.

Self-propelled capsules or tablets introduced into and against the flow of blood have been used for years to deliver coagulant active pharmaceutical ingredients to the site of the bleeding wound, for example (Baylis et al. Sci. Adv. 1:e1500379, 2015). The concept is simple and elegant: gas, produced as a result of contact between the capsule's reactive contents and the fluid in which it is introduced, is produced and vigorously propels the capsule's contents toward the wound and vasculature, against the flow of blood. Similarly, APIs have been administered orally and bioavailability, enhanced in the GI system through sustained release and floating approaches through a variety of techniques, e.g., as effervescent tablets, involving the release or production of gas, permitting the APIs to be absorbed into circulation and transported to their intended site of action. (Kamalakkannan et al. Intl. J. Drug Delivery 3:558-70, 2011)

Further, APIs in the form of pharmaceutical compositions have been administered using medical devices to position the compositions (e.g., in various forms: particles, powders, gels, liquids and solids) into sites difficult to access without a more invasive surgical procedure. In the case of the inner ear of the human, medical devices may be used for delivery of the composition through one device such as a catheter and, during the same intervention, other procedures such as implantation of a cochlear implant or electroporation of tissues for uptake of pharmaceutical compositions, for example.

As noted, it can be advantageous to deliver therapeutic substances to the body of a recipient, including various body chambers. However, using conventional techniques, it is difficult to deliver therapeutic substances to certain body chambers (e.g., fluidically-sealed chambers) within the body of a recipient without compromising the near-term or long-term structural and functional integrity of certain structures within the chamber. For example, it can be advantageous to deliver therapeutic substances to the apical region of the cochlea for various reasons. The apical region of the cochlea is, however, extremely difficult (i) to access physically without damaging sensitive tissues, and (ii) to treat either locally or systemically with pharmaceutical preparations.

As such, presented herein are techniques configured to deliver therapeutic substances, in an atraumatic manner, to a chamber within the body of a recipient (e.g., a “body chamber”). As described elsewhere herein, the techniques presented herein can be used to deliver therapeutic substances to any of a number of body chambers located, for example, behind a number of different tissue barriers, including, fluidically-sealed body chambers located behind the blood-brain barrier (BBB), behind the blood-labyrinth barrier (BLB), behind the blood-ocular barrier (BOB), which includes the blood-aqueous barrier (BAB) and the blood-retinal barrier (BRB), and so on. For example, the techniques presented herein can be used to deliver therapeutic substances the scala tympani, the scala media, the scala vestibuli, the semi-circular canals, any other volume of the labyrinthine, the retina, etc.

More specifically, presented herein are embodiments for pharmaceutical compositions encapsulated in self-propelling capsules or tablets (“pharmaceutical delivery capsules” or “capsules”), and their methods of administration of such compositions in fluid-containing body chambers/compartments. Pharmaceutical delivery capsules, for example, may be delivered to the base of the cochlea, where the capsule is then propelled by gas released by it within the cochlea fluid for self-propelled transport away from the base of the cochlea (e.g., towards the apical region of the cochlea). As the capsule is transported through a region of the cochlea, the therapeutic substance(s) contained in the capsule are distributed within the fluid and exert their effects on surrounding tissues. This method of administration is advantageous in the preparation of such tissues for implantation of a medical device such as a cochlear implant, and alternatively, may be used immediately prior to or after implantation of a medical device such as a cochlear implant. Similarly, method embodiments disclosed herein may be used to deliver therapeutic substances solely for treatment of distally located tissues bordering a fluid-containing chamber, such as a tissue barrier, or to other difficult to reach tissues within the cranium and other areas of the body.

Preparation of tissues for implantation comprises the administration of one or more anti-inflammatory APIs to reduce CI electrode implantation-associated damage to tissues in the region of implantation. In some embodiments the preparation of tissues for implantation involves administration of one or more APIs, which prepare the tissue for one or more additional APIs to be administered prior to the initiation of surgery. In some embodiments, an API is administered within 3 weeks, within 2 weeks, within 1 week, within 6 days, within 5 days, within 4 days, within 3 days within 2 days, within 1 day, within 12 hours, within 6 hours, within 3 hours, within 2 hours within 1 hour, within 30 minutes, within 15 minutes within 10 minutes, or between 2 and 15 minutes, between about 5 and 30 minutes, between about 10 and 35 minutes, between 15 and 60 minutes prior to beginning implantation or other surgery. In some preferred embodiments, the API is administered within 30 minutes of beginning surgery. In other preferred embodiments, one or more APIS are administered 1 hour to 7 days prior to beginning surgery. In some embodiments, APIs such as dexamethasone are administered prophylactically at a dose of about 40 ng/ml; in other embodiments the APIs may be administered between about 1 ng/mL and about 1 mg/mL with the goal of distributing the one or more APIs throughout the fluid-containing chambers.

The self-propelled capsule embodiment, in addition to other embodiments disclosed herein, are especially useful for distributing therapeutic substances in fluid-filled (i.e., fluid-containing) chambers of the body in which limited fluid movement occurs, such as, without limitation: (i) the bony labyrinth, the osseous labyrinth, and the otic capsule, which include the perilymph and endolymph fluid chambers of the vestibular and cochlear system; (ii) the anterior, posterior, and vitreous chambers in the eye; (iii) the CSF filled spaces including the subarachnoid space (between the arachnoid mater and the pia mater), the ventricular system (around and inside the brain and spinal cord), the ventricles of the brain, cisterns and sulci, as well as the central canal of the spinal cord; (iv) the pericardial cavity (serous fluid-filled space between the heart and pericardial sac) and (v) abscesses.

Merely for ease of description, the techniques presented herein will primarily be described with reference to implantable delivery of therapeutic substances to a specific area/cavity of a recipient, namely the cochlea of a recipient. However, as noted, it is to be appreciated that the techniques presented herein can be used to deliver therapeutic substances such as pharmaceutical compositions to other areas within the body of a recipient, whether human or another animal species.

It is also to be appreciated that the techniques presented herein may also be implemented alone or in combination with a number of different types of implantable medical devices. For example, the techniques presented herein may be implemented by auditory prosthesis systems that include one or more other types of auditory prostheses, such as middle ear auditory prostheses, bone conduction devices, direct acoustic stimulators, electro-acoustic prostheses, auditory brain stimulators, cochlear implants, combinations or variations thereof, etc. The techniques presented herein may also be used with tinnitus therapy devices, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

### Cochlear Anatomy

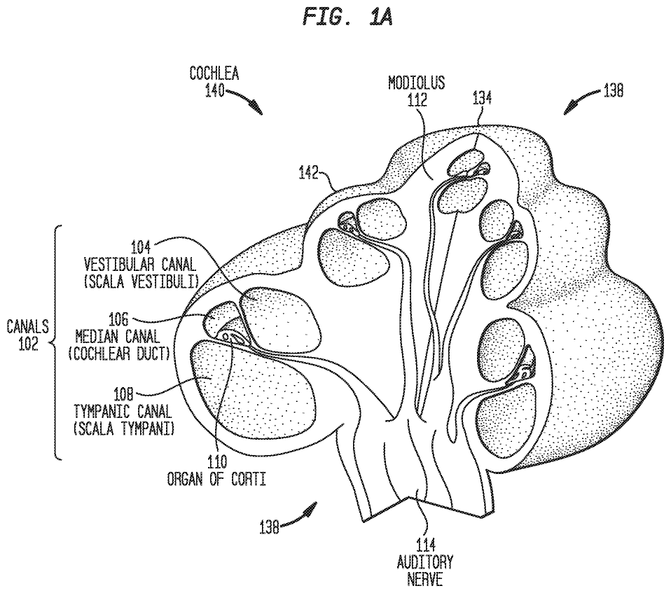

Relevant aspects of an example cochlea 140 are described below with reference to FIGS. 1A-1B. More specifically, FIG. 1A is a perspective view of the cochlea 140 partially cut-away to display the canals and nerve fibers of the cochlea, while FIG. 1B is a cross-sectional view of one turn of the canals of the cochlea 140.

Referring first to FIG. 1A, cochlea 140 is a conical spiral structure comprising three parallel fluid-containing canals or ducts, collectively and generally referred to herein as canals 102. Canals 102 comprise the tympanic canal 108, also referred to as the scala tympani 108, the vestibular canal 104, also referred to as the scala vestibuli 104, and the median canal 106, also referred to as the scala media 106. Cochlea 140 spirals about modiolus 112 several times and terminates at cochlea apex 134.

Portions of cochlea 140 are encased in a bony labyrinth/capsule 116 and the endosteum 121 (e.g., a thin vascular membrane of connective tissue that lines the inner surface of the bony tissue that forms the medullary cavity of the bony labyrinth). Spiral ganglion cells 114 reside on the opposing medial side 120 (the left side as illustrated in FIG. 1B) of cochlea 140. A spiral ligament membrane 130 is located between lateral side 118 of spiral tympani 108 and bony capsule 116, and between lateral side 118 of scala media 106 and bony capsule 116. Spiral ligament 130 also typically extends around at least a portion of lateral side 118 of scala vestibuli 104.

The fluid in the tympanic canal 108 and the vestibular canal 104, referred to as perilymph, has different properties than that of the fluid which fills scala media 106 and which surrounds organ of Corti 110, referred to as endolymph. The tympanic canal 108 and the vestibular canal 104 collectively form the perilymphatic fluid space 109 of the cochlea 140. Sound entering a recipient's auricle (not shown) causes pressure changes in cochlea 140 to travel through the fluid-containing tympanic and vestibular canals 108, 104. As noted, the organ of Corti 110 is situated on basilar membrane 124 in the scala media 106 and contains rows of 16,000-20,000 hair cells (not shown) which protrude from its surface. Above them is the tectoral membrane 132 which moves in response to pressure variations in the fluid-containing tympanic and vestibular canals 108, 104. Small relative movements of the layers of membrane 132 are sufficient to cause the hair cells in the endolymph to move thereby causing the creation of a voltage pulse or action potential which travels along the associated nerve fiber 128. Nerve fibers 128, embedded within the spiral lamina 122, connect the hair cells with the spiral ganglion cells 114 which form auditory nerve 114. Auditory nerve 114 relays the impulses to the auditory areas of the brain (not shown) for processing.

The place along basilar membrane 124 where maximum excitation of the hair cells occurs determines the perception of pitch and loudness according to the place theory. Due to this anatomical arrangement, cochlea 140 has characteristically been referred to as being “tonotopically mapped.” That is, regions of cochlea 140 toward basal region 136 are responsive to high frequency signals, while regions of cochlea 140 toward apical region 138 are responsive to low frequency signals. These tonotopical properties of cochlea 140 are exploited in a cochlear implant by delivering stimulation signals within a predetermined frequency range to a region of the cochlea that is most sensitive to that particular frequency range.

In general, the basal region 136 is the portion of the cochlea 140 located closest to the stapes (not shown in FIGS. 1A and 1B) and extends to approximately the first turn of the cochlea (i.e., the region of the cochlea 140 between the cochlea openings, including the round and oval windows, the first cochlea turn). The apical region 138 is portion of the cochlea 140 in proximity to the cochlear apex 134. More specifically, the cochlea 140 is generally a conical spiral structure (i.e., the spiral-like shape) and the apical region 138 of the cochlea 140 is generally the last/final (i.e., most apical) 360 degrees of the cochlea and encompasses the cochlea areas tonotopically associated with frequencies below 1000 Hz.

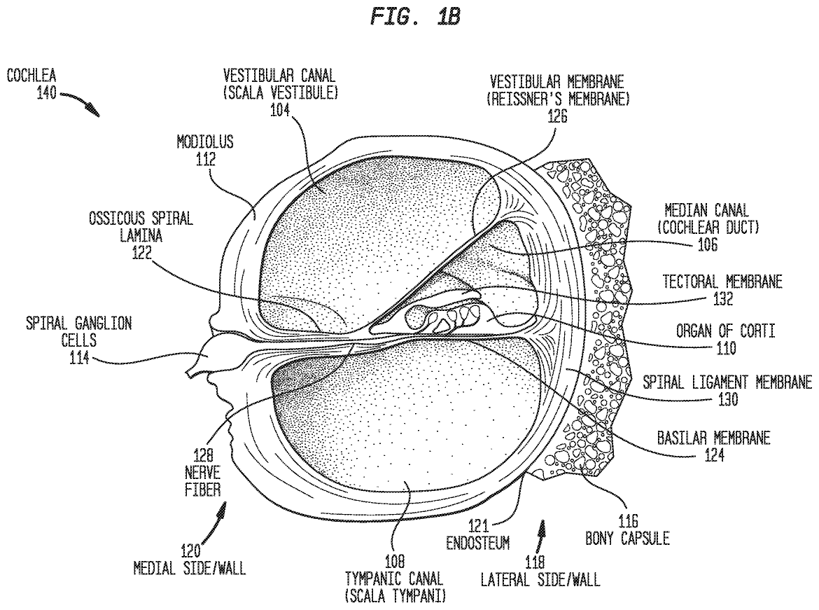

FIG. 2 is a schematic diagram depicts one embodiment of a self-propelled capsule 250, in accordance with certain embodiments presented herein. More specifically, FIG. 2 illustrates that the self-propelled capsule 250 comprises an outer shell/housing 252 forming two chambers 254 and 256, separated by a wall 258. The first chamber 254, sometimes referred to herein as a “therapeutic substance chamber,” includes one or more therapeutic substances 264 therein. The second chamber 256, sometimes referred to herein as “propulsion chamber,” includes a propulsion system 266 therein (e.g., a propulsion system within a capsule). In this example, the propulsion system 266 is configured to, upon exposure to body fluid (e.g., perilymph), produce a gas 265 that is released via one or more openings 268 in the propulsion chamber 256. As shown, the one or more openings 268 are located on one side of the capsule 252 such, upon release of the gas, the self-propelled capsule 250 will be “propelled” (e.g., forced to move) in a direction that is generally opposite to the direction in which the gas is released (e.g., in a direction that is generally opposite to the one or more openings 268). Stated differently, the production and release of gas on one side (or from a particular portion) of the capsule 250 results in propulsion of the capsule in a direction that is generally opposite that of the release of gas.

As noted, the gas is produced and released when the propulsion system 266 is exposed to bodily fluid. The bodily fluid can enter the propulsion chamber 256 via, for example, the one or more openings 268, via pores in the housing 252, etc. In certain embodiments, the housing 252 can be bioresorbable.

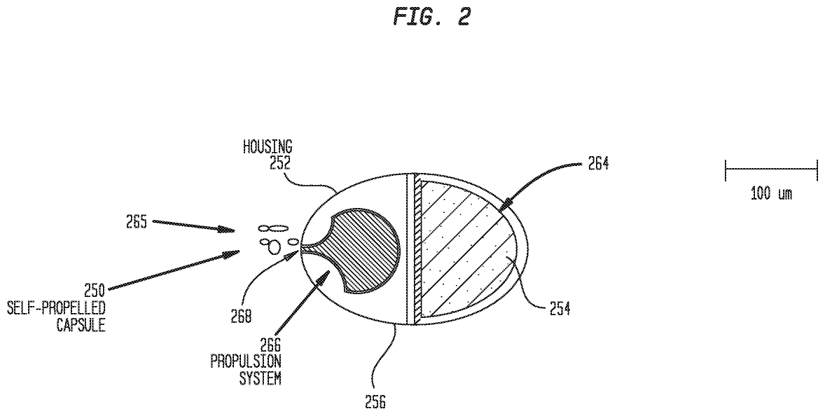

As noted, FIG. 2 illustrates an embodiment in which the self-propelled capsule 250 includes two chambers that separate the propulsion system from the therapeutic substances to be delivered to the recipient. In alternative embodiments, the propulsion system and the therapeutic substances are not separated. For example, FIG. 3 depicts another embodiment in which the propulsion system is uniformly distributed throughout the capsule or throughout a majority portion of the capsule (e.g., equal or greater than 75% of the volume of the core of the capsule).

More specifically, FIG. 3 is a schematic diagram depicts one embodiment of a self-propelled capsule 350, in accordance with certain embodiments presented herein. The self-propelled capsule 350 comprises an outer shell/housing 352 forming a single chamber 354, that includes a propulsion system 366 therein (e.g., a propulsion system within a capsule) and one or more therapeutic substances 364. Similar to the embodiment of FIG. 2, the propulsion system 366 is configured to, upon exposure to body fluid (e.g., perilymph), produce a gas 365 that is released via one or more openings 368 in the housing 352. As shown, the one or more openings 368 are located on one side of the capsule 352 such, upon release of the gas, the self-propelled capsule 350 will be “propelled” (e.g., forced to move) in a direction that is generally opposite to the direction in which the gas is released (e.g., in a direction that is generally opposite to the one or more openings 368). Stated differently, the production and release of gas on one side (or from a particular portion) of the capsule 350 results in propulsion of the capsule in a direction that is generally opposite that of the release of gas.

As noted, the gas is produced and released when the propulsion system 366 is exposed to bodily fluid. The bodily fluid can enter the chamber 354 via, for example, the one or more openings 368, via pores in the housing 352, etc. In certain embodiments, the housing 352 can be bioresorbable.

In the example of FIG. 3, the propulsion system 366 may also result in the capsule being propelled to a particular region of the fluid-containing chamber. However, this arrangement can also result in uniform distribution of the pharmaceutical composition (and the therapeutic substance(s) (APIs) contained therein) throughout the fluid-containing chamber.

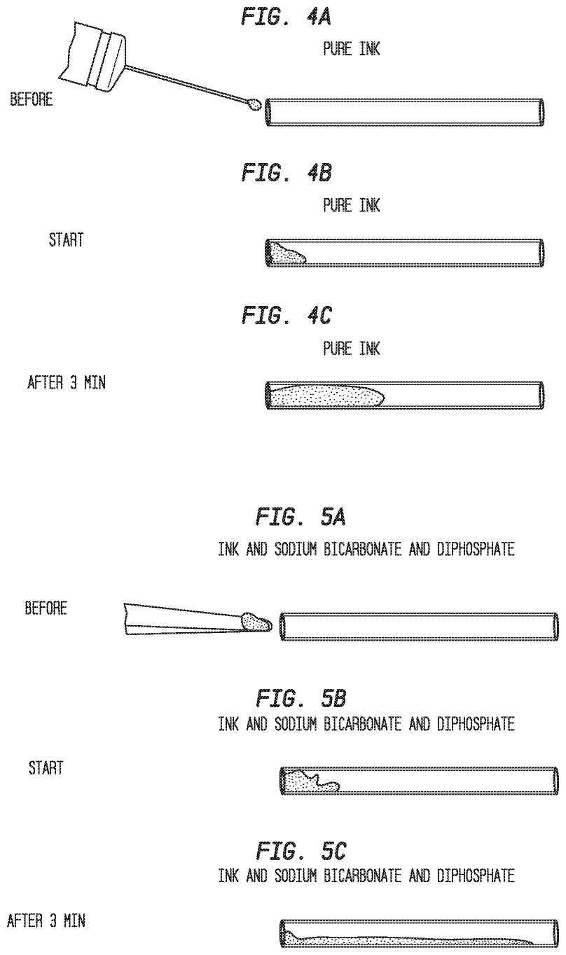

In other embodiments the core of the capsule and its outer coating are one in the same material, i.e., there is no separate coating enclosing the API(s) and propulsion system: the capsule. In some embodiments the capsule is materially uniform throughout. (FIGS. 5A-5C show this one embodiment in its simplest form.) In other embodiments the capsule is materially uniform except for air or other case pockets contained within the bodt. To prepart a materially uniform capsule, in one embodiment, the sodium bicarbonate (or mixture comprising it, diphosphate and starch) is adhered to or coated on one or more APIs; in another embodiment, one or more of sodium bicarbonate, diphosphate or starch is adhered to or coated on the one or more APIs while other components are admixed with the APIs and formed into a tablet. In other embodiments the mixture may be administered in powder form. Upon contact with a bodily fluid such as perilymph, the components dissolve and the APIs are propelled randomly within the fluid-containing chamber.

In still other embodiments the capsule contains a solid, poreless coating to allow for easier packaging and shipping and a longer shelf-life. At the time of administration, the capsule coating is pierced with a needle or other sharp object that is a part of the device used to deliver and/or place the capsule in the fluid-containing chamber. In other embodiments to coating contains pores to permit gas expulsion.

In still other embodiments, two or more capsules are administered simultaneously into the same fluid-containing chamber. In other embodiments two or more APIs are encompassed within a single coated or uncoated capsule.

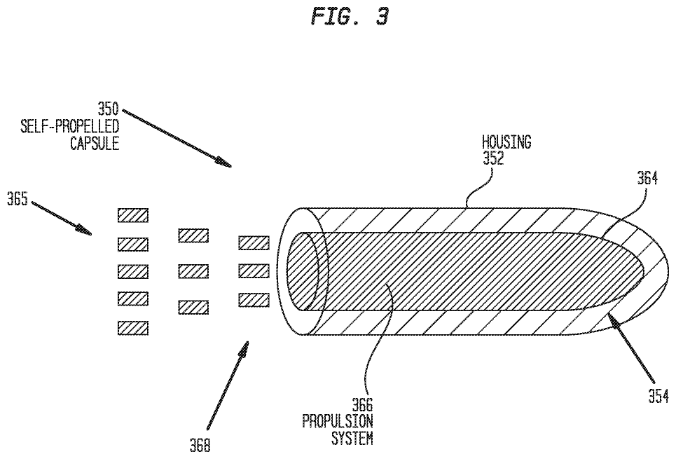

FIGS. 4A, 4B, and 4C are a series of photographs taken before, immediately after (at the start), and three minutes following the addition of blue ink at one end of a saline (0.9% sodium chloride solution) filled, 2 mm long polyurethane tube. The series of photographs (FIGS. 4A-4C) show that the blue ink travelled about one third or 7 mm of the length of the tube through saline by passive diffusion within 3 minutes.

FIGS. 5A, 5B, and 5C are series of photographs depicting the same experiment with ink combined with a propellant (sodium bicarbonate, diphosphate and corn starch in powder form), before the release of the mixture, upon release (the start), and again three minutes later. FIGS. 5A-5C show that in the presence of a propellent (sodium bicarbonate) the ink has travelled the full length of the tube though saline in 3 minutes. This experiment demonstrated that the addition of a propellent lead to a more rapid (3×) distribution through the saline-containing tube.

### Pharmaceutical Compositions and their Preparation

Single compounds or molecules as well as the combinations of active and nonactive compounds including biologically active molecules and compounds, together with all other components that may be required to formulate a particular form (tablet, powder, liquid, gel, etc.) for a particular mode of administration to deliver a therapeutically effective amount are collectively and generally referred to in this disclosure as “pharmaceutical compositions.” The term “compound” or “agent” may also refer to any single component that comprises a pharmaceutical composition. Pharmaceutical compositions or “compositions,” as that term is used, are administered to treat disorders or diseases of the tissues bordering a fluid in a chamber, reaching to more distal tissues beyond that border. Compositions, e.g., may modulate properties of the brain labyrinth barrier and, specifically may act to reduce inflammation and/or otherwise modulate the permeability of the brain labyrinth barrier and its surrounding tissues. If inflammation is the result of injury at the site of implantation of a medical device such as a cochlear implant, in preferred method embodiments, the attenuation of increasing inflammation over the days that ensue is a primary goal.

In some embodiments one pharmaceutical composition containing one or more therapeutic substances (e.g., APIs) may be administered, while in other embodiments two or more therapeutic substances may be combined and administered simultaneously or in series. Examples of therapeutic substances include but are not limited to large and small molecule therapeutic substances, viral vectors or almost any other type of known therapeutic substances, since the effect of the therapeutic substance is not dependent on crossing, e.g., from the stomach into the blood. Rather, administration is local and direct into a target tissue, such as the fluid-containing chamber of the cochlear canal.

Therapeutic substances may take any appropriate form or media in the disclosed embodiments including, capsules, tablets, powders, particles (e.g., nanoparticles) or slow-release compositions such as those available commercially. In all embodiments the size of the particles or capsules should not exceed the diameter of available cochlear implant electrodes or other therapeutic substance delivery apparatus for accessing fluid-containing chambers in the body, e.g., 0.5 mm.

In the disclosed embodiments, pharmaceutical compositions may comprise any single or combination of the following therapeutic substances: biological substances, bioactive substances, conjugated or fusion molecules or compounds, viral and non-viral vectors, natural, synthetic and recombinant molecules, antibodies and antibody fragments, etc., pharmaceutical agents/active pharmaceutical ingredients (APIs) including commercially available versions of the same, genes, nucleases, endonucleases, nucleic and ribonucleic acids such as messenger RNA (mRNA), siRNA and miRNA, naked DNA, DNA vectors, oligonucleotides, antisense polynucleotides, peptides, polypeptides, proteins including binding proteins, anti-oxidants, and signalling compounds that promote recovery and resolution, other chemicals, ions, and other molecules used to modulate inflammation within the body of individual. A person of ordinary skill in the art will appreciate that each if these substances can be generated by methods known in the art.

Small molecule therapeutic substances (APIs) include, without limitation, steroids (e.g., dexamethasone, triamcinolone, fluticasone, prednisolone), antibiotics (including aminoglycoside antibiotics, e.g., Kanamycin, Gentamicin), antiapoptotics, antioxidants, antihistamines, NSAID (non-steroidal anti-inflammatoires), N-Methyl-D-aspartate (NMDA) receptor antagonists (for treating Tinnitus), therapeutic substance combinations (e.g., FX-322), GSK-3 inhibitors, Wnt activators, sodium thiosulfate (for treating cisplatin-associated ototoxicity, nephrotoxicity and neurotoxicity).

Large molecule therapeutic substances include, without limitation, protein-based therapeutics (therapeutic proteins) including peptides, recombinant proteins, monoclonal antibodies and vaccines, antibody-based therapeutic substances, Fc fusion proteins and other conjugated molecules, anticoagulants, blood factors, bone morphogenetic proteins, engineered protein scaffolds, enzymes, growth factors, hormones including neurotrophins, interferons, interleukins, and thrombolytics.

(API) compounds that are currently in an experimental state include: antioxidants such as HPN-07 and NAC; anesthetics; neurotrophins, mRNA and AAV based gene therapies such as otoferlin and human atonal transcription factors (ATOH1) cDNA, Gamma secreatase inhibitors, JNK stress kinase inhibitors, Kv3 positive modulators; nucleophiles such as sodium thiosulfate pentahydrate to bind to ototoxic compounds such as cisplatin, urea-thiophene carboxamide, 5-HT3 receptor antagonists (e.g., azasetron besylate); ebselen, D-Methionine, Lantanoprost, Xalatan, neurotrophic factors (e.g., BDNF, NT3), Zonasamide and Dendrogenin.

In some embodiments disclosed, one or more pharmaceutical compositions are administered to address the integrity of a tissue barrier, including, among others: (i) vasoconstrictors (e.g., alpha-adrenoceptor agonists, vasopressin analogs, epinephrine, norepinephrine, phenylephrine (Sudafed PE), dopamine, dobutamine, migraine and headache medications (serotonin 5-hydroxytryptamine agonists or triptans)); (ii) corticosteroids (e.g., dexamethasone, bethamethasone, (Celestone), prednisone (Prednisone Intensol), prednisolone (Orapred, Prelone), triamcinolone or triamcinolone-acetonide (Aristospan Intra-Articular, Aristospan Intralesional, Kenalog), and methylprednisolone (Medrol, Depo-Medrol, Solu-Medrol)); (iii) compounds or molecules that have or produce anti-pneumolysin activity to address bacterial-mediated disruption of the brain labyrinth barrier; and (iv) compounds or molecules that have or produce anti caveolin-1 activity, blocking caveolin-1 receptors, or suppressing caveolin-1 over-expression. In preferred embodiments, the pharmaceutical composition comprises compounds from two or more of these four categories.

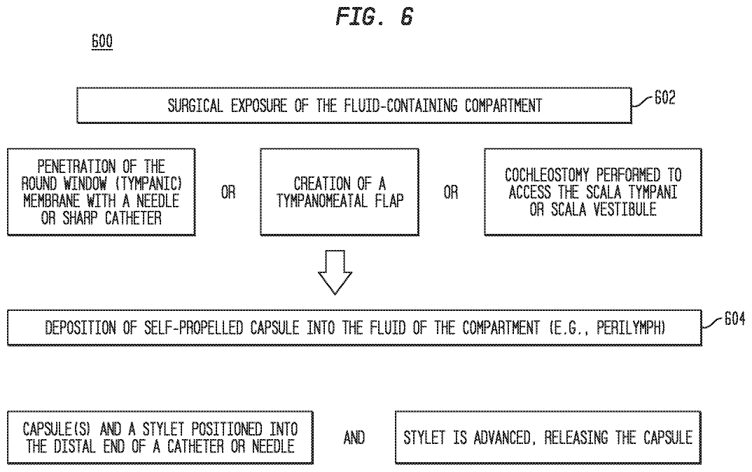

FIG. 6 is a flowchart 600 depicting the general process for administering a therapeutic substance as part of a pharmaceutical composition in a fluid-containing chamber. In FIG. 6, at 602, a fluid-filled/fluid-containing body compartment/chamber is surgical exposed. The fluid-containing body chamber can be exposed in a number of different manners that are appropriate for the specific chamber. For example, in the context of a recipient's inner ear, the surgical exposure can occur via penetration of the round window (tympanic) membrane with a needle or sharp catheter, via creation of a tympanomeatal flap, via cochleostomy performed to access the scala tympani or scala vestibule, etc.

At 604, a self-propelled capsule is deposited into the fluid of the chamber (e.g., perilymph). Again, the self-propelled capsule can be deposited in a number of different manners, such as via a stylet positioned into the distal end of a catheter or needle where the stylet is advanced, thereby releasing the capsule.

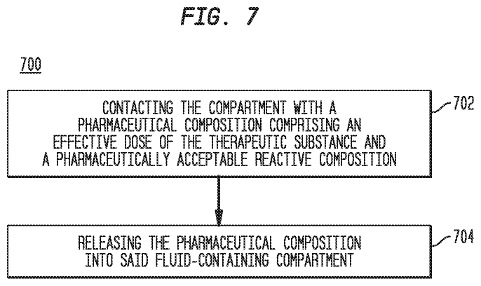

FIG. 7 is a flowchart of an example method 700, in accordance with embodiments presented herein. Method 700 begins at 702 where a body compartment/chamber is contacted with a pharmaceutical composition comprising an effective dose of the therapeutic substance and a pharmaceutically acceptable reactive composition. At 704, the pharmaceutical composition into the fluid-containing chamber. The pharmaceutically acceptable reactive composition generates a gas upon contact with a bodily fluid in the fluid-containing chamber, and wherein the gas causes fluidic movement within the fluid-containing chamber and propulsion of the therapeutic substance, leading to distribution in the fluid and contact of the therapeutic substance with barrier tissues of the fluid-containing chamber.

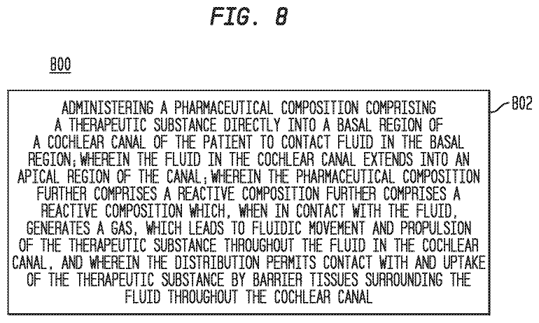

FIG. 8 is a flowchart of an example method 800, in accordance with embodiments presented herein. Method 800 begins at 802 where a pharmaceutical composition comprising a therapeutic substance is administered directly into a basal region of a cochlear canal of the patient to contact fluid in the basal region. The fluid in the cochlear canal extends into an apical region of the canal, the pharmaceutical composition further comprises a reactive composition which, when in contact with the fluid, generates a gas, which leads to fluidic movement and propulsion of the therapeutic substance throughout the fluid in the cochlear canal, and the distribution permits contact with and uptake of the therapeutic substance by barrier tissues surrounding the fluid throughout the cochlear canal.

In certain embodiments disclosed, one or more pharmaceutical compositions are administered, e.g., to treat a particular condition, disorder or disease, or to modulate properties of a tissue barrier, for example. In some of these embodiments, e.g., the brain labyrinth barrier is deliberately disrupted or destabilized by administration of the composition. Examples of such destabilizing compositions include, among others: salicylate, lipopolysaccharide (LPS), keyhole limpet hemocyanin (KLH), and a variety of vestibular-active molecules, such as anticholinergics and antihistamines, as well as other membrane destabilizing proteins or peptides known in the art (see, e.g., Fernandez et al., 2009). For example, LPS has been shown to induce systemic inflammation, compromising the integrity of the barrier (Hirose et al., 2014).

A person of ordinary skill in the art of pharmaceuticals will appreciate that the prodrug form of any of the above-listed therapeutic substances (APIs) may be used for any of the disclosed embodiments as may be necessary to prepare one or more pharmaceutical compositions and/or use such compositions in the method embodiments disclosed.

Conjugated molecules are, as the name suggests, molecules linked together to form a complex, which can be administered for treating a wide variety of disorders and diseases. These molecules are characterized by having a cell-permeable (or cell-penetrating) component that facilitates delivery of another, linked component, a molecule or compound with biological activity, to intracellular or intranuclear sites of action where they may elicit a variety of effects. (See, e.g., Kristensen and Nielsen. Tissue Barriers, 4:2, e1178369, DOI:10.1080/21688370.2016.1178369.) These include, e.g., among other things, the regulation of gene expression through interference with post-transcription processes. Some conjugated molecules have been used for facilitated transport of bioactive molecules across tissue barriers and into cells and may be particularly important as components of a pharmaceutical compositions for the embodiments disclosed herein for transporting molecules from the fluid of the chamber into tissue barrier cells lining the chamber.

Numerous types of vectors can be used to deliver and express one or more therapeutic molecules in target cells. In some embodiments, viral vectors such as adenovirus (Ad) vectors, adeno-associated virus (AAV) vectors, retrovirus vectors, herpesvirus, and other viral vectors; likewise, nonviral vectors may be used, e.g., naked DNA, oligonucleotides, lipolexes, nanoparticles and sleeping beauty. APIs in cell therapies may further be used in some embodiments, e.g., NT cells such as those available from LCT Global (lctglobal.com).

Small interfering RNA (siRNA) molecules are a prime example of an active molecule that can be delivered, as a complex with a vector, to a target cell. While the high molecular weight and negative charge of double-stranded siRNA molecules would prevent them from crossing the brain labyrinth barrier, siRNA molecules have been coupled to vectors to facilitate transport to sites difficult to access. In one instance, labeled siRNA was delivered to inner ear cells by coupling it to a non-viral vector to facilitate transport to sites difficult to access (Qi et al., 0214). These conjugated molecules have the potential to treat a variety of disorders and diseases by targeting particular cells and blocking the production of particular molecules, e.g., those involved in inflammation or cancer. siRNA molecules have been used successfully to modulate the brain labyrinth barrier by interfering with the production of connexin 43, an important protein constituent of the tight junctions between endothelial cells of the brain labyrinth barrier, and thus integrity of the tissue barrier (Zhang et al., 2020). siRNA molecules further have been used in intra-cochlear gene therapy research in mice to target allele suppression to slow the progression of hearing loss (Yoshimura et al., 2019).

In certain aspects, disorders of tissue barriers may be addressed. For example, inhibition of the expression of inflammatory cytokines can be achieved by administration of pharmaceutical compositions comprising inhibitory nucleic acids (e.g., dsRNAs, siRNAs, antisense oligonucleotides, etc.) directed to inhibit cytokine expression or activity. In some embodiments, pharmaceutical compositions comprise siRNA molecules coupled with transporter proteins or other molecules to facilitate entry from the perilymph or endolymph fluids, or other fluids, into cells and nuclei. In one embodiment the transporter protein (coupled to a siRNA molecule) is one that is recognized by tissue barrier cells, permitting entry of the siRNA or other molecule into these cells. In other embodiments the pharmaceutical compositions comprise one or more siRNA or other oligonucleotides. In yet other embodiments, the pharmaceutical composition comprises one or more conjugated molecules. In some method embodiments, such pharmaceutical compositions are prepared and administered to modulate properties of the cells of the tissue barrier, to block protein or peptide production or for another effect.

In certain method embodiments, the concentration of inflammatory cytokines is decreased by administering pharmaceutical compositions comprising a corticosteroid such as dexamethasone. Such compositions may be systemically administered in an amount sufficient to give a final dexamethasone concentration in the perilymph of at least about 20 ng/ml to up to 1 mg/mL, depending on the mode of administration, for example, between about 20 nM to about 1200 nM, from about 10 nM to about 35 nM, from about 35 to about 45 nM, from about 45 nM to about 100 nM, from about 50 nM to about 250 nM, from about 200 nM to about 300 nM, from about 225 nM to about 400 nM, from about 250 nm to about 500 nM, from about 400 nM to about 600 nM, from about 500 nM to about 750 nM, from about 550 nM to about 850 nM, from about 650 nM to about 900 nM, or from about 700 nM to about 1.0 mg (Yixu Wang et al. A comparison of systemic and local dexamethasone administration: From perilymph/cochlea concentration to cochlear distribution, Hearing Res., 370:1-10, 2018. ISSN 0378-5955, https://doi.org/10.1016/j.heares.2018.09.002).

In certain method embodiments, expression of inflammatory cytokines at the tissue barrier is decreased by systemically administering pharmaceutical compositions comprising one or more compounds having or producing anti-pneumolysin activity. Such compositions may be systemically administered in an amount sufficient to give a final composition concentration in the perilymph in the range from about 20 nM to about 1200 nM, from about 10 nM to about 35 nM, from about 35 to about 45 nM, about 40 nM, from about 45 nM to about 100 nM, from about 50 nM to about 250 nM, from about 200 nM to about 300 nM, from about 225 nM to about 400 nM, from about 250 nm to about 500 nM, from about 400 nM to about 600 nM, from about 500 nM to about 750 nM, from about 550 nM to about 850 nM, from about 650 nM to about 900 nM, or from about 700 nM to about 1.2 mg. In some embodiments such compositions are administered systemically; in other embodiments the compositions are administered locally to the middle or inner ear using methods described herein.

In certain other method embodiments, expression of inflammatory cytokines is decreased by administering pharmaceutical compositions comprising one or more compounds having or producing anti-caveolin-1 activity. Such compositions may be systemically administered in an amount sufficient to give a final composition concentration in the perilymph in the range from about 20 nM to about 1200 nM, from about 10 nM to about 50 nM, from about 25 nM to about 100 nM, from about 50 nM to about 250 nM, from about 200 nM to about 300 nM, from about 225 nM to about 400 nM, from about 250 nm to about 500 nM, from about 400 nM to about 600 nM, from about 500 nM to about 750 nM, from about 550 nM to about 850 nM, from about 650 nM to about 900 nM, or from about 700 nM to about 1.2 mg. In some embodiments such compositions are administered systemically; in other embodiments the compositions are administered locally to the middle or inner ear using methods described herein.

In some embodiments of pharmaceutical compositions, an siRNA molecule may be coupled with a transport molecule for targeting and suppressing caveolin-1 overexpression and, thus, in embodiments of methods incorporating such pharmaceutical compositions for modulation of the brain labyrinth barrier, it may reduce the transport of molecules from the blood through cells of the barrier. In still other embodiments pharmaceutical compositions comprising siRNA molecules are administered target the expression of immune system actors such as TNF-α, IL-1β and other cytokines responsible for the immune response to injury or irritation, i.e., those having anti-pneumolysin activity. In some pharmaceutical composition embodiments, the siRNA molecules may be protected from degradation by being packaged in known non-viral nano-particle-based carrier systems, or encased in polymers, silica, porous silicon or lipids, for example (Kim et al., 2019). Pharmaceutical composition embodiments may be combined with other such embodiments to be implemented in one or more disclosed method embodiments to modulate tissue barrier properties.

Further, in some embodiments known gene editing technology may be used to excise or replace sections of genes that, e.g., encode regulators or cytokine availability in any of the tissues of the inner ear, including the brain labyrinth barrier and its surrounding tissues. For example, in some embodiments, gene editing strategies employing the various technologies known in the art, including but not limited to the CRISPR/cas9 system, among others, are used to correct genetic disorders to the extent such disorders manifest as permeable brain labyrinth barrier (and other barrier) malfunctions. A person of ordinary skill in the art would appreciate that other gene editing technologies known in the art may be used in such embodiments, and other Cas or other enzymes, proteins or peptides may be functional in the Cas9 role. Gene editing methods known in the art can be performed upon the cells of a subject in vivo (or ex vivo and then administered as a component of a pharmaceutical composition in the disclosed embodiments). Stem cell therapies may further be used to generate components of pharmaceutical composition embodiments.

In some embodiments of pharmaceutical compositions, those comprising one or more compounds having or producing anti-caveolin-1 activity and/or anti-pneumolysin activity are effective for modulating the permeability and possibly other properties to improve brain labyrinth barrier integrity. In other method and pharmaceutical composition embodiments, combinations comprising any two or more of corticosteroids, vasoconstrictors and compounds having or producing anti-caveolin-1 activity and/or anti-pneumolysin activity in any form may be administered simultaneously or in serial by any single or combination of modes of administration to modulate the permeability of the brain labyrinth barrier. Such embodiments may be used as a part of treatment regimens involving monitoring and preventing inflammation due to the increased expression of cytokines at or near the brain labyrinth barrier and resulting loss of its integrity.

To prepare the pharmaceutical compositions according to the disclosed embodiments, a therapeutically effective amount of one or more of the compounds or compositions according to the disclosed embodiments are preferably intimately admixed with a pharmaceutically acceptable carrier, diluent or excipient, according to conventional pharmaceutical compounding techniques to produce a dose. The term “pharmaceutically acceptable carrier diluent or excipient” refers to any substance, not itself a therapeutic agent, used as a carrier or vehicle, or non-active component of the composition for administration to an individual, or added to a pharmaceutical composition to improve its handling or storage properties, or to permit or facilitate formation of a unit dose of the composition, and that does not produce unacceptable toxicity or interaction with other components in the composition.

The amount of composition included within therapeutically active compositions according to the disclosed embodiments is an effective amount for affecting the desired outcome, e.g., in the tissues of the cochlear apex, bordering the fluid-containing chamber into which the composition is released.

The choice of pharmaceutically acceptable carrier, excipient or diluent may be selected based on the composition and the intended route of administration, as well as standard pharmaceutical practice. Such compositions may comprise any agents that may aid, regulate, release, or increase entry into the body chamber/compartment, tissue, intracellular or intranuclear target site, such as binder(s), lubricant(s), suspending agent(s), coating agent(s), solubilizing agent(s), or other agents. Non-limiting examples include polymers and silicones. Administration of an implant of a composition for the sustained release may also be used to obtain prolonged exposure and action, and in some embodiments may be, e.g., a liquid, gel, or solid implant or may be in the form of particles, including nanoparticles. The term “sustained release” refers to compositions from which the composition is released at a slow rate allowing for a longer period of exposure at active concentrations.

A carrier, as well as other components in the composition, may take a wide variety of forms depending on the form of preparation desired for administration, e.g., including solutions, gels, films, particles, powders, creams, (some of which may be further formulated as sustained release preparations).

In some embodiments the pharmaceutical composition comprises one or more therapeutic substances (APIs) mixed with the propelling agent (a reactive composition), such as sodium bicarbonate and diphosphate, each in powder or particulate form, and is administered as a powder. In other embodiments the composition is in the form of a liquid or gel and is administered in that form, rather than as pressed into a tablet or capsule. Once the composition, regardless of form—tablet, powder, particulate, gel or liquid—comes into contact with the body fluid, the composition undergoes a phase change to gas, resulting in further distribution of the therapeutic substance in the composition in the fluid due to fluidic movement and propulsion of the therapeutic substance. If the composition is in the form of a capsule, it is propelled in the fluid as gas is released and it dissolves, and the therapeutic substance (API) is released.

In some embodiments the pharmaceutical composition is an injectable, water-free suspension of liquid therapeutic substances (APIs) mixed with a propellant in particulate or crystal form. Unlike in embodiments in which the propellant in particle form is adhered to the therapeutic substance, the liquid (or gel, for example) therapeutic substance in this suspension is not adhered to the particulate reactive composition. When these particles come into contact with the bodily fluid of the fluid-containing chamber, gas is produced, which creates a motion in the fluid, distributing the liquid API in the fluid.

In some embodiments the propelling agent is a reactive composition, which is a pharmaceutically acceptable carbonate or hydrogen carbonate and a pharmaceutically acceptable acid. In the disclosed embodiments the propellant comprises a combination of a carbonate or bicarbonate, in addition to one or more pyrophosphates or one or more organic acids such as citric, tartaric, fumaric, malic, adipic acids, as well as anhydrides and salts of the acid. In some embodiments, potassium, sodium or arginine carbonate (or bicarbonate) can be used as alkalis (an alkali metal or alkaline earth metal salt). Sodium bicarbonate is particularly soluble, reacts well and is cost effective. The carbonate and acid are independently soluble in the aqueous fluid of the chamber, and upon contact with each other a reaction takes place, generating carbon dioxide.

In some embodiments in which a coating is applied to the therapeutic substance in capsule or tablet form, the coating materials used to prepare the coating may be degradable in the body. Standard API encapsulation techniques may be used (as is used for taste masking or preparing a slow release coating for orally administered tablets, e.g., to produce core-shell type capsules). For example, the API and propellant are mixed and then encapsulated in a core-shell type capsule. Finally, gas-releasing channels or pores are created as described below. (For further information, see, e.g., K-S Seo, et al. Pharmaceutical applications of tablet film coating. Pharmaceutics 2020, 12, 853; doi:103390/pharmaceutics12090853; and P. Yin Yee Chin, et al. A review of in vivo and in vitro real-time corrosion monitoring system of biodegradable metal implants. Applied Sci. 2020, 10, 3141, doi:10.3390/a10093141.) Additional information on biodegradable polymers can be found at: https://healthcare.evonik.com/en/medical-devices/biodegradable-materials/resomer-portfolio/standard-polymers. See also techniques on application of the coating at: https://lubrizolcdmo.com/technical-briefs/encapsulation/#spray-processes. In some embodiments a permeable or semi-permeable coating encapsulates the table or capsule, permitting aqueous fluid to come into contact with the core of the capsule comprising a reactive composition. The coating layer may be biodegradable in the body; in other embodiments the coating layer may be enzymatically degraded to permit the fluid to reach the reactive composition of the capsule.

In certain embodiments the channels or pores that release gas are generated by known methods including without limitation the following.
generating channels in vivo with a two-phase membrane comprising particles and polymer, in which particles, reacting with the perilymph or other fluid, dissolve faster than the polymer, creating channels for gas to escape;
generating channels in vivo using propellant gas pressure. Here the membrane is permeable to water, which reacts with the propellant and forms gas inside the capsule. The pressure increases in the capsule until it bursts, releasing gas. The thickness of the membrane (uniformly or in certain portions) as well as the choice of polymer are two parameters that can be modified to achieve the desired gas release profile;
generating channels ex vivo using traditional processes for porous membrane fabrication, such as interfacial polymerization, or the simplest option: phase inversion, which can be achieved through non-solvent-induced phase separation (NIPS), or evaporation-, vapor-, or thermally induced phase separation; and
generating channels mechanically ex vivo using laser ablation of the membrane, e.g., as described in Yu, et al. In situ separation of chemical reaction systems based on a special wettable PTFE membrane. Adv Functional Mat. December 2017, DOI:10.1002/adfm.201703970.
generating one or many channels in vivo using mechanical means such as piercing the membrane with a needle or sharp catheter which is part of the applicator tool at the time of administration.

In some situations, greater buoyancy of the capsule may be desired, meaning that at 37 degrees Celsius (body temperature), the capsule is less dense than perilymph fluid. In some embodiments the capsule is made to be buoyant through increasing porosity, which decreases its density. (See e.g., Kamalakkannan et al. Intl J Pharm Appl. 3:558-70-94, 2011; Jagdale et al. Intl J Pharm Appl. 2(3):181-94, 2011.) Porosity can be adjusted by controlling the density of the materials used in its production, e.g., flexibility of the polymer membrane or coating or the type of propellant (reactive composition). The porosity of the therapeutic substance/propellant mix can be titrated through changes in particle size and/or compression force when capsules are formed. In some embodiments the ratio of the API to the propellant and/or coating material may also modulate porosity. In certain embodiments the diameter of the channels or pores that release gas may be adjusted to result in a tighter (i.e., less “leaky”) capsule upon coming into contact with a body fluid; eventually, pressure building inside the capsule due to gas production causes the capsule to expand, which opens the pores to the point that the gas produced equals that released; gas release (and buoyancy) is also modulated through membrane or coating thickness. In some embodiments the amount of gas produced and trapped inside the capsule may be adjusted based on pore size, polymer flexibility and membrane thickness, changing the buoyancy of the capsule. The porosity affects not only the density of the capsule, but also the speed at which the capsule dissolves in contact with body fluids. Accordingly, for greater buoyancy and a faster rate of dissolution, a relatively high level of porosity can be achieved when producing capsules. The volume of propellant is calculated to keep the capsule buoyant and propelled through the fluid to achieve a homogenous and continuous distribution of the therapeutic substance load. The rate of therapeutic substance release as a capsule dissolves can also be slowed depending on materials used in its production.

In some embodiments the capsules may be prepared with more or less air or other gas trapped inside the capsule core or under a coating layer to make the capsule (or tablet) less dense and therefore, buoyant in the fluid. In still other embodiments, the capsule may comprise a material more dense than water or than the fluid in the fluid-containing chamber. In certain embodiments this dense material may be iron oxide.

In some embodiments the capsule is less dense than perilymph at body temperature. In other embodiments, the capsule has the same density as perilymph. In still other embodiments the capsule is denser than perilymph at body temperature. In still other embodiments, the capsule has been designed for limited gas production so that after having been delivered to the fluid-containing chamber such as the perilymph of the inner ear, it is buoyant for a time until there is no more gas produced and expelled, at which time the fluid begins to fill the channels of the capsule, making it denser and permitting it to sink while it continues to dissolve and release the therapeutic substance load. In some embodiments, the capsules contain small barb-like structures on their surface that anchor them into the tissues against which they have settled.

Once the fluid has come into contact with the reactive composition, and depending on the materials incorporated into it, pressure builds from the gas produced and one or more channels allow its escape, propelling the capsule within the fluid-containing chamber. If the pharmaceutical composition is in the form of a capsule, its size must be sufficiently small to be able to navigate the in increasingly smaller areas of the fluid-containing chamber, such as the cochlear canal, moving from base to apex.

In some embodiments the reactive composition is located on a first side of a capsule and the pharmaceutical composition is located on a second side of the capsule, resulting in gas being generated and propelling the capsule in a direction opposite to that of the direction gas is expelled. In other embodiments, the pharmaceutical composition and reactive composition are mixed and, when produced, gas is expelled from all surfaces of the capsule, propelling the capsule.

In some embodiments, the active compound is included in the pharmaceutically acceptable carrier or diluent in an amount sufficient to deliver to a recipient a therapeutically effective amount for the desired indication, without causing serious toxic effects or being cleared from the system before it can reach its intended site of action.

### Modes of Administration

Approaches to the delivery of pharmaceutical compositions include, for the embodiments disclosed herein, localized administration/delivery approaches where the pharmaceutical compositions are initially delivered at or near target locations within the recipient. The goal of localized delivery is that the target location, and possibly a small amount of surrounding tissue, is exposed to the pharmaceutical composition. In some embodiments the pharmaceutical compositions is distributed widely within a localized, fluid-containing chamber to which the composition (capsule, powder, etc.) is delivered. In other embodiments the pharmaceutical compositions may be delivered and the therapeutic substance load released in phases, first, throughout the chamber as the capsule is propelled in the fluid, and later, in a more concentrated release pattern in a particular area of the fluid-containing chamber, such as when a capsule no longer produces gas and sinks to the bottom of the chamber where it continues to release the therapeutic substance load in that region of the chamber. In certain embodiments, the pharmaceutical compositions is distributed uniformly in a fluid-containing chamber of the inner ear, including into the apex of the cochlea. Thus, pharmaceutical compositions are delivered at the specific target location and may remain in a relative proximity to the target delivery location. One example of localized delivery is catheterizing the femoral artery (in the thigh) and guiding the catheter to a specific location, distal to the insertion point, where a pharmaceutical composition is released to act locally, such as in the arterial supply to the cochlea, or other vasculature in the brain tissue.

In the disclosed embodiments, a pharmaceutical composition is administered locally and invasively into a closed, fluid-containing chamber where the composition, comprising a reactive composition, and having been administered in almost any form, comes into contact with the fluid of that chamber and produces gas. The gas propels the composition, also comprising therapeutic substance(s), to other areas of the chamber and surrounding tissues where the therapeutic substance(s) can be further transported in these tissues. If the composition is delivered in the form of a capsule, the capsule is propelled as it dissolves and releases the therapeutic substance load within the fluid of the chamber. If the composition is in the form of a powder, similarly, the gas produced distributes the therapeutic substance(s) throughout the chamber In certain embodiments, the composition is delivered into the cochlear canal where it comes into contact with fluid of the inner ear, e.g., perilymph or endolymph. Gas is produced, propelling the capsule as it disintegrates with in the fluid, distributing the therapeutic substance (API) to tissues bordering the fluid and, importantly, into the apical region of the cochlear canal.

In some embodiments, the pharmaceutical composition may be introduced into the body via an outlet of the catheter or through the implantation of a medical device, which is positioned at the target location within the recipient. In other embodiments, the pharmaceutical composition is delivered some time before an implantation procedure to prepare the tissues, e.g., to attenuate an immune response due to potential damage to the tissues during the implantation. This period before implantation may be less than S minutes prior, between about 5 to 30 minutes prior, about 30 to 60 minutes prior, about 60 to 90 minutes prior, about 90 minutes to about 2 hours prior, about 2 to 4 hours prior, about 4 to 6 hours prior, about 6 to 10 hours prior, about 10 to 16 hours prior.

In other embodiments, delivery approaches may include intra-cochlear delivery of the pharmaceutical composition immediately preceding, or simultaneously with, introduction of a cochlear implant electrode array, or by injection into the cochlea.

In some embodiments pharmaceutical compositions may be delivered into the basal region of the cochlear canal, while other therapeutic substances are administered concurrently, locally into the arterial system serving structures of the inner ear, or systemically. In other embodiments pharmaceutical compositions may be delivered to fluid-containing chambers in the cranium for treatment of distal tissues.

Some types of localized administration suffer from the problem that certain areas of a recipient's body are difficult to access in a manner that allows for the direct delivery of the pharmaceutical composition. The disclosed embodiments, however, address the issue of treating tissues that are otherwise unavailable during the usual modes of local administration by taking advantage of the production of gas and its effect to propel a dissolving capsule, e.g., to a region even further distal, where its contents will be distributed for the intended effect.

In the context of the inner ear, localized administration of a therapeutic substance to the base of the cochlea is invasive but does not pose the risk of toxicity as systemic administration does; nonetheless, the apex of the cochlear canal is difficult to treat either locally or systemically, and is physically difficult to reach with a medical device without causing damage to the tissues, particularly the hair cells, which are involved in hearing. For example, injecting a pharmaceutical composition into the cochlea may cause the loss of residual hearing due to disruption of the brain labyrinth barrier, e.g., through destruction of hair cells, forming an opening the cochlea that changes the cochlear dynamics, and other effects.

In some embodiments, administration of a pharmaceutical composition to the perilymph or endolymph fluid chambers may include any of the following known methods: (i) via injection or deposition of the composition at the round window membrane, or through a cochleostomy to the scala tympani; (ii) through a direct cochleostomy to the scala media; (iii) using a combination of those two pathways; and (iv) implanting a cochlear implant or other medical device with therapeutic substance-eluting electrodes (pharmaceutical compositions comprising a reactive composition contained in a coating through a polymer, silica, silicone or other coating that is permeable or semi-permeable to enable contact between the fluid and the electrode), or delivered through a separate delivery cannula associated with the electrode, or other similar delivery channel) (Wang et al., 2018).

A person of ordinary skill will appreciate the impact pharmacokinetics of the therapeutic substance(s) administered in the disclosed embodiments and how the pharmacokinetics may affect the dose, the timing, and duration of treatment. Several excellent reviews and discussions are available regarding local (middle/inner ear) pharmacokinetics of various pharmaceutical agents (see, e.g., Plontke et al., 2017; Salt 2005; Nyberg et al 2019; Salt and Plontke 2018; Rybak et al., 2019).

Some of the methods presented herein are primarily described with reference to a specific implantable medical device system, namely, a cochlear implant system. However, a person of ordinary skill in the art would appreciate that the methods disclosed also may be used with other types of implantable medical devices or implantable medical device systems. For example, the methods presented herein may be used with other auditory prosthesis systems and related devices. The methods and pharmaceutical compositions disclosed herein may also be used with tinnitus therapy devices, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, and others.

### Doses and Schedules

One of ordinary skill in the art will recognize that in the disclosed embodiments a therapeutically effective amount provided in the composition, as well as the dosing, and timing of dosing will depend on the condition being treated and its severity, the pharmaceutical composition and its pharmacokinetics, the mode of administration, and other factors such as weight and condition of the patient, and the judgment of the prescribing caregiver, among other considerations.

In general, a therapeutically effective amount of the pharmaceutical compositions in dosage form usually ranges from less than about 0.001 mg/kg patient body weight to about 2.5 g/kg patient body weight on a per hour, day or other time period basis, regardless if those amounts are delivered in a single dose or apportioned over multiple periods of administration in the specified period. In the most preferred embodiments, pharmaceutical compositions according to disclosed embodiments are administered in a suitable carrier in amounts ranging from about 1 mg/kg to about 100 mg/kg per hour, day or per other period, again, regardless if those amounts are delivered in a single dose or apportioned over multiple periods of administration in the specified period.

In the disclosed, preferred embodiments, a therapeutically effective amount of the pharmaceutical composition, i.e., comprising one or more APIs in dosage form, depends on the APIs, the type of excipients and/or carriers, the elution rate, the mode and location of administration, and the duration of treatment period and schedule, e.g. a regular or irregular dosing period. Typically, a physician or other caregiver licensed to prescribe pharmaceuticals will determine the actual dosage most suitable for an individual subject.

In the disclosed, preferred embodiments, a therapeutically effective amount administered in the cochlea or other fluid-containing chamber is usually less than 0.1 mg/kg body weight, less than about 0.01 mg/kg body weight, less than about 0.001 mg/kg body weight, less than about 0.0001 mg/kg body weight, less than about 0.00001 mg/kg, less than about 0.000001 mg/kg body weight, ranges between about 0.000001 mg/kg to about 0.00001 mg/kg body weight, ranges between about 0.00001 mg/kg to about 0.0001 mg/kg, ranges between about 0.0001 mg/kg to about 0.001 mg/kg body weight, and ranges between about 0.01 mg/kg to about 1.0 mg/kg.

All ranges and ratios discussed here can and necessarily do describe all values, subranges and subratios therein for all embodiments, and all such subranges and subratios also form part and parcel of the disclosed embodiments. Any listed range or ratio can be easily recognized as sufficiently describing and enabling the same range or ratio being broken down into at least equal halves, thirds, quarters, fifths, tenths, etc. and each range or ratio discussed herein can be readily broken down into a lower third, middle third and upper third, etc. Alternatively, a range of 1 to 50 is understood to include any number, fraction or combination of numbers from the group of positive numbers between about 0 and up to and including about 50.

In some embodiments two or more pharmaceutical compositions may be administered simultaneously; in other embodiments two or more pharmaceutical compositions may be administered serially, e.g., when one or more APIs are administered in one or more pharmaceutical compositions to prepare the tissue for receipt of one or more subsequent APIs. In still other embodiments two or more pharmaceutical compositions, whether in the form described in the embodiments herein, can each be administered through the same or a different mode of administration (e.g., systemic, intra-cochlear, locally to the middle ear).

In certain method embodiments, the pharmaceutical composition is administered once daily; in other embodiments, the compound is administered twice to six times daily; in yet other embodiments, the compound is administered once every two days, once every three days, once every four days, once every five days, once every six days, once every seven days, once every two weeks, once every three weeks, once every four weeks, once every two months, once every six months, or once per year. In other embodiments, the composition is administered on an irregular basis. In still other embodiments, the composition is administered on an as-needed basis.

In some embodiments, methods comprise the pharmaceutical composition being administered for modulating the permeability of the brain labyrinth barrier, which administration will extend for time periods of about 1 to 24 hours, 1 to 4 days, 3 to 6 days, 5 to 8 days, exceeding one week, two weeks, three weeks, one month, two months, three months, four months, five months, six months, 9 months, one year, two years, three years, four years, or five years, ten years, or fifteen years; or for example, any time period range in hours, days, months or years in which the low end of the range is any time period between 1 hour and 2 years, 2 to 10 years, 15 years, and the upper end of the range is between 15 days and 20 years (e.g., between 4 weeks and 15 years, between 6 months and 20 years). In some cases, it may be advantageous for the compositions of the disclosed embodiments and their modifications to be administered for the life of the patient.

Otic conditions typically are treated by administering multiple doses of drops or injections over several days and up to two weeks, sometimes with multiple doses administered daily. Pharmaceutical compositions of the embodiments disclosed herein may be re-administered at any desired frequency (e.g., daily, weekly, etc.) to achieve a suitable therapeutic effect. In some embodiments, the composition may be delivered prior to or simultaneous with a cochlear implant or during the cochlear implantation procedure. In other embodiments, the compositions may be delivered with any other type of middle or inner ear implant or device, or as a component of a known control system.

A person of ordinary skill in the art will appreciate that the dosing interval may need to be adjusted according to the needs of individual patients. In some embodiments involving longer intervals of administration, compositions may be administered in a form appropriate for sustained release, including the formation of depots for such release.

In preferred embodiments, the administration of pharmaceutical compositions remains effective for at least 12 hours, at least 1 day, at least 3 days, at least 1 week, two weeks, three weeks, one month, two months, three months, four months, five months, six months, one year, two years, three years, four years, or five years, ten years, fifteen years, twenty years, or for the remainder of the subject's life.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

1-57. (canceled)

58. A method comprising:
administering a pharmaceutical composition comprising a therapeutic substance directly into a basal region of a cochlear of a patient to contact fluid in the basal region;
wherein the pharmaceutical composition further comprises a reactive composition which, when in contact with the fluid, generates a gas, which leads to fluidic movement and propulsion of the therapeutic substance throughout the fluid in the cochlear canal.

59. The method of claim 58, wherein the pharmaceutical composition is a water-free suspension.

60. The method of claim 58, wherein the therapeutic substance is selected from a group consisting of: steroids (such as dexamethasone, triamcinolone, fluticasone and prednisolone), antibiotics, antiapoptotics, antioxidants, antihistamines, NTHEs, NMDA receptor agonists, GSK-3 inhibitors, Wnt activators, sodium thiosulfate, protein-based therapeutics, antibodies, vaccines, antibody-based therapeutics, fusion proteins, conjugated molecules, nucleic acids, anticoagulants, blood factors, bone morphogenetic proteins, engineered protein scaffolds, enzymes, growth factors, hormones, mRNA-and AAV-based gene therapies, gamma secreatase inhibitors, JNK stress kinase inhibitors, Kv3 positive modulators, nucleophiles, 5-HT3 receptor antagonists, zonasamide, and dendrogenin.

61. The method of claim 58, wherein the therapeutic substance comprises a corticosteroid.

62. The method of claim 58, wherein the method comprises administering the pharmaceutical composition to a perilymphatic space of the cochlea.

63. The method of claim 58, wherein the therapeutic substance comprises a conjugated molecule comprising an siRNA, miRNA, shRNA or other nucleic acid molecule, peptide or protein.

64. The method of claim 58, wherein the pharmaceutical composition is a sustained release composition.

65. The method of claim 58, wherein the method comprises administering the therapeutic substance to prepare tissue of the recipient for cochlear implant implantation, and subsequently implantable a cochlear implant.

66. The method of claim 65, wherein the pharmaceutical composition is administered between about 5 to 30 minutes prior to implantation of the cochlear implant.

67. The method of claims 58, wherein the gas produced causes fluidic movement within the chamber and propels the therapeutic substance to apical region of the cochlear.

68. The method of claim 58, wherein the method comprises modifying the permeability of a tissue barrier bordering the fluid in the cochlear, and administering the pharmaceutical composition to the fluid in the basal region of the cochlea across the tissue barrier.

69. A method of administering a therapeutic substance to a fluid-containing chamber of a body, comprising:
contacting the chamber with a pharmaceutical composition comprising an effective dose of the therapeutic substance and a pharmaceutically acceptable reactive composition, wherein the pharmaceutical composition is in a liquid or a gel form, and
releasing the pharmaceutical composition into the fluid-containing chamber;
wherein the pharmaceutically acceptable reactive composition generates a gas upon contact with a bodily fluid in the fluid-containing chamber, and
wherein the gas causes fluidic movement within the fluid-containing chamber and propulsion of the therapeutic substance, leading to distribution in the fluid and contact of the therapeutic substance with barrier tissues of the fluid-containing chamber.

70. The method of claim 69, wherein the pharmaceutical composition is contacted with the fluid-containing chamber by administering the pharmaceutical composition by using a medical device.

71. The method of claim 70, wherein the medical device is a cochlear implant.

72. The method of claim 69, wherein the pharmaceutical composition is a water-free suspension.

73. The method of claim 69, wherein the therapeutic substance is selected from a group consisting of: steroids (such as dexamethasone, triamcinolone, fluticasone and prednisolone), antibiotics, antiapoptotics, antioxidants, antihistamines, NTHEs, NMDA receptor agonists, GSK-3 inhibitors, Wnt activators, sodium thiosulfate, protein-based therapeutics, antibodies, vaccines, antibody-based therapeutics, fusion proteins, conjugated molecules, nucleic acids, anticoagulants, blood factors, bone morphogenetic proteins, engineered protein scaffolds, enzymes, growth factors, hormones, mRNA-and AAV-based gene therapies, gamma secreatase inhibitors, JNK stress kinase inhibitors, Kv3 positive modulators, nucleophiles, 5-HT3 receptor antagonists, zonasamide, and dendrogenin.

74. The method of claim 69, wherein the therapeutic substance comprises a corticosteroid.

75. The method of claim 74, wherein the corticosteroid is dexamethasone.

76. The method of claim 69, wherein the therapeutic substance comprises a conjugated molecule comprising an siRNA, miRNA, shRNA or other nucleic acid molecule, peptide or protein.

77. The method of claim 69, wherein the pharmaceutical composition is a sustained release composition.

78. The method of claim 69, wherein the fluid-containing chamber is selected from the group consisting of: the bony labyrinth, the osseous labyrinth, the otic capsule, the cochlear canal, and the vestibular system of the ear; the anterior or posterior vitreous chamber of the eye; a chamber containing cerebrospinal fluid; the pericardial cavity; and an abscess.

79. The method of claim 69, wherein in preparing tissues for implantation of a medical device the pharmaceutical composition is delivered to the fluid-containing chamber between about 40 minutes and 7 days prior to implantation of the medical device.

80. The method of claim 79, wherein one or more additional therapeutic substances is administered between about 5 and 30 minutes prior to implantation of the medical device.

81. The method of claim 69, wherein in preparing tissues for implantation of a medical device, the pharmaceutical composition is delivered to the fluid-containing chamber between about 5 and 30 minutes prior to implantation of the medical device.

82. The method of claim 69, wherein the therapeutic substance is delivered to a basal region of the cochlear canal, and wherein the gas causes fluidic motion and also propels or distributes the therapeutic substance in the bodily fluid to an apical region of the cochlear canal.

83. A pharmaceutical composition configured for delivery of a therapeutic substance to a fluid-containing chamber of a body of a recipient, comprising:
an effective dose of the therapeutic substance; and
a pharmaceutically acceptable reactive composition;
wherein the reactive composition generates a gas upon contact with a bodily fluid in the fluid-containing chamber;
wherein the generation of the gas upon contact with the pharmaceutically acceptable reactive composition with a bodily fluid creates motion in the fluid-containing chamber, and propels or causes distribution of the therapeutic substance throughout the fluid-containing chamber,
wherein the pharmaceutical composition is administered in liquid or gel form.
